(19) United States
(12) Patent Application Publication
(10) Pub. No.: US 2010/0106218 A1
(43) Pub. Date: Apr. 29, 2010

(54) CONFIGURATION OF A STIMULATION MEDICAL IMPLANT

(75) Inventor: Andrew Botros, Maroubra (AU)

Correspondence Address: CONNOLLY BOVE LODGE & HUTZ LLP, 1875 EYE STREET, N.W., SUITE 1100, WASHINGTON, DC, 20006, US

(73) Assignee: COCHLEAR LIMITED, Lane Cove, NSW (AU)

(21) Appl. No.: 12/441,273

(22) PCT Filed: Sep. 14, 2007
(86) PCT No.: PCT/AU2007/001369

§ 371 Date: Sep. 21, 2009

(30) Foreign Application Priority Data
Sep. 14, 2006 (AU) 2006905072

(51) Int. Cl. A61F 11/04; A61N 1/36

(52) U.S. Cl. 607/57

(57) ABSTRACT

A method for configuring a medical implant that stimulates a physiological system of a recipient of the medical implant is described where the medical implant includes at least one input configuration variable corresponding to a subjective characteristic of the physiological system. The configuration method includes measuring at least one physical characteristic of the physiological system to provide at least one objective physical measurement value and then determining the at least one input configuration variable to configure the medical implant by a predictive configuration model having as an input the at least one objective physical measurement value.

### CROSS REFERENCE TO RELATED APPLICATIONS

This application is a National Stage of PCT/AU2007/01369 which claims priority from Australian Provisional Patent Application No. 2006905072 entitled “Medical Implant Configuration Method”, filed 14 Sep. 2006, which are hereby incorporated by reference herein.

### BACKGROUND

1. Field of the Invention

The present invention relates generally to medical implants that stimulate a physiological system, and more particularly, to the configuration of stimulation medical implants.

2. Related Art

There are many medical implants that deliver electrical stimulation to a recipient for a variety of therapeutic benefits. Cochlear implants, such as those manufactured under the brand name Cochlear™ for example, have been developed to provide persons with sensorineural hearing loss with the ability to perceive sound. The hair cells of the cochlea of a normal healthy ear converts acoustic signals into nerve impulses. People who are profoundly deaf due to the absence or destruction of cochlea hair cells are unable to derive suitable benefit from conventional hearing aid systems. Cochlear implants have been developed to provide such persons with the ability to perceive sound.

Cochlear implants typically comprise external and implanted or internal components that cooperate with each other to provide sound sensations to the recipient. The external component traditionally includes a microphone or other sound input component that detects sounds, such as speech and environmental sounds, a speech processor that selects and converts certain detected sounds, particularly speech, into a coded signal, a power source such as a battery, and an external transmitter antenna.

The coded signal output by the speech processor is transmitted transcutaneously to an implanted receiver/stimulator unit. This transcutaneous transmission occurs via the external transmitter antenna which is positioned to communicate with an implanted receiver antenna disposed within the receiver/stimulator unit. This communication transmits the coded sound signal while also providing power to the implanted receiver/stimulator unit.

The implanted receiver/stimulator unit also includes a stimulator that processes the coded signal and outputs an electrical stimulation signal to an intra-cochlear electrode assembly. The electrode assembly typically has a plurality of electrodes that apply electrical stimulation to the auditory nerve to produce a hearing sensation corresponding to the original detected sound.

Following surgical implantation of the internal components (including the receiver/stimulator unit and intra-cochlear electrode assembly), the cochlear implant system must be configured (or fitted) for each individual recipient. This configuration procedure is normally carried out by an audiologist, clinician or other healthcare professional several weeks after implantation.

An important aspect of this configuration procedure is the collection and determination of a number of recipient-specific input configuration variables that are required for normal operation of the cochlear implant system. Typically, these input configuration variables include a threshold level of electrical stimulation (known as a T level), and a maximum comfort level of electrical stimulation (known as a C level) for each electrode stimulation channel. Together, the T and C levels define a “dynamic range” of electrical stimulation for each electrode channel.

Conventionally, T and C levels are manually determined by the clinician working together with the recipient. For each electrode channel of the implant, the clinician applies stimulation pulses, and then receives an indication from the recipient, as to the level and comfort of the resulting sound.

The T level is defined as the level at which the recipient first identifies sound sensation, and is the lowest level which causes a hearing percept in the recipient.

The C level sets the maximum allowable stimulation level for each electrode and is defined as the maximum stimulation level that does not produce an uncomfortable loudness sensation for the recipient.

It is desirable, for optimum perception of sound and speech by the recipient, that the dynamic range be correctly configured. If a T level is too low, then stimuli are applied which cannot be perceived. If the C level is too high, then the recipient may be overstimulated, leading to pain and possible injury.

It should be stressed in relation to determining T and C levels in this way, that it is not so much important that the T or C levels conform precisely to a psychophysical definition. Rather, the important factor is how well the recipient hears and understands detected speech or sounds.

This post-operative configuration process has been extremely time consuming. In locations where there is a lack of adequate audiological infrastructure and/or trained clinicians, a cochlear implant may not be optimally fitted for each particular recipient. Additionally, since this post-operative configuration process relies on subjective measurements, children, pre-lingually deaf or congenitally deaf patients are often unable to provide an accurate impression of the resultant hearing sensation resulting from the stimulation test pulses. This further complicates the process, potentially resulting in a cochlear implant that is not optimally fitted.

### SUMMARY

In one aspect the present invention there is provided a method for configuring a medical implant that stimulates a physiological system of a recipient of the medical implant, the medical implant having at least one input configuration variable corresponding to a subjective characteristic of the physiological system. The method comprises measuring at least one physical characteristic of the physiological system to provide at least one objective physical measurement value; and determining the at least one input configuration variable to configure the medical implant by a predictive configuration model having as an input the at least one objective physical measurement value.

In another aspect the present invention there is provided a medical implant system for the stimulation of a physiological system of a recipient of the medical implant, the medical implant system having at least one input configuration variable corresponding to a subjectively determined characteristic of the physiological system, the medical implant system comprises measurement means to measure at least one physical characteristic of the physiological system to provide at least one objective physical measurement value; and data processing means for processing the at least one corresponding objective physical measurement value according to a predictive configuration model to provide the at least one input configuration variable to configure the medical implant system.

### BRIEF DESCRIPTION OF THE DRAWINGS

Exemplary embodiments of the present invention are described below with reference to the accompanying drawings, in which:

FIG. 1 is an exemplary cochlear implant system which may be advantageously implemented with embodiments of the present invention;

FIG. 2A is a flowchart of a configuration method according to an exemplary embodiment of the present invention;

FIG. 2B is a schematic data flow diagram used to further explain the method of FIG. 2A;

FIG. 3 is a system overview of a machine learning algorithm as employed in an exemplary embodiment of the present invention to provide a predictive configuration model, as shown in FIG. 2B;

FIG. 4 is a table depicting data used to train the machine learning algorithm illustrated in FIG. 3, in accordance with one embodiment of the present invention;

FIG. 5 is a graph of predicted T levels predictive configuration model versus measured T levels;

FIG. 6 is a graph of predicted C levels generated by a predictive configuration model versus measured C levels;

FIG. 7 is a flowchart depicting how the Cubist machine learning algorithm constructs a predictive configuration model according to an exemplary embodiment of the present invention; and

FIG. 8 is a block diagram of a cochlear implant system arranged to be able to implement a configuration method according to an exemplary embodiment of the present invention.

### DETAILED DESCRIPTION

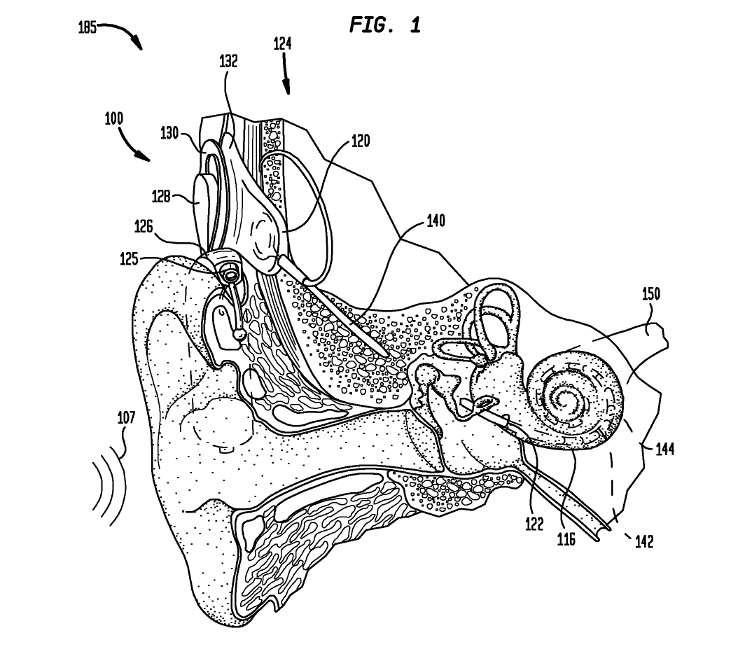

Referring to FIG. 1, a cochlear implant system 185 comprises external component assembly 100 and internal (or implanted) component assembly 124. External assembly 100 comprises a behind the ear (BTE) speech processing unit 126 connected to a transmission coil 130. The BTE unit includes a microphone 125 for detecting sound which is then processed by electronics within the BTE unit to generate coded signals. The coded signals are provided to an external transmitter unit 128, along with power from a power source such as a battery (not shown).

Internal component assembly 124 includes a receiver unit 132 having an internal coil (not shown) that receives and transmits power and coded signals from external assembly 100 to a stimulator unit 120 to apply the coded signal along an electrode assembly 140. Electrode assembly 140 enters cochlea 116 at cochleostomy region 122 and has one or more electrodes 142 positioned to be substantially aligned with portions of cochlea 116.

Cochlea 116 is tonotopically mapped with each region of the cochlea being responsive to acoustic and/or stimulus signals in a particular frequency range. To accommodate this property of cochlea 116, the cochlear implant system 185 includes an array 144 of electrodes each constructed and arranged to deliver suitable stimulating signals to particular regions of the cochlea, each representing a different frequency component of a received audio signal 107. Signals generated by stimulator unit 120 are applied by electrodes 142 of electrode array 144 to cochlea 116, thereby stimulating the auditory nerve 150. It should be appreciated that although in FIG. 1 electrodes 142 are arranged in an array 144, other arrangements and other types of contacts are possible.

Typically, electrode array 144 includes a plurality of independent electrodes 142 each of which can be independently stimulated. As one of ordinary skill in the art is aware, low frequency sounds stimulate the basilar membrane most significantly at its apex, while higher frequencies more strongly stimulate the basilar membrane's base. Thus, electrodes 142 of electrode array 144 located near the base of cochlea 116 are used to simulate high frequency sounds while electrodes closer to the apex are used to simulate lower frequency sounds.

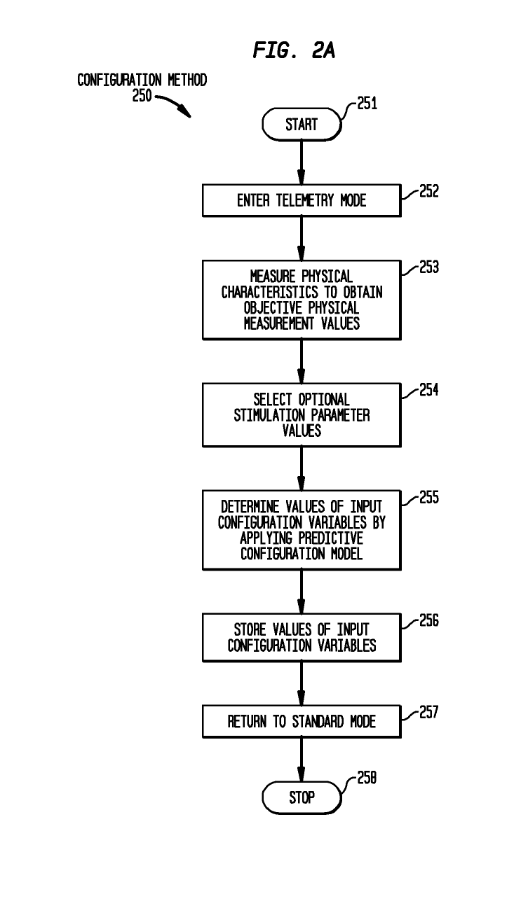

Further details of the above and other exemplary cochlear implant systems in/with which embodiments of the present invention may be implemented include, but are not limited to, systems described in U.S. Pat. Nos. 4,532,930, 6,537,200, 6,565,503, 6,575,894 and 6,697,674, U.S. Pat. No. 5,758,651, WO 2005/122887, each of which are hereby incorporated by reference herein. FIG. 2A is a flowchart of an exemplary embodiment of a configuration method of the present invention. FIG. 2B is a schematic data flow diagram illustrating with the flow of data during the performance of the configuration method in FIG. 2A. Configuration method 250 may be performed intra-operatively and/or post-operatively, to determine values of a number of recipient-specific input configuration variables that are required for normal operation of a cochlear implant such as cochlear implant system 185.

Configuration method 250 commences at block 251, followed immediately by block 252 in which a telemetry mode of cochlear implant system 185 is enabled.

The telemetry mode enables a telemetry facility within cochlear implant system 185 to measure various physical characteristics of the recipient's physiological system. In telemetry mode, implanted electrode array 144 is used to provide test stimuli, and to then measure a neural response of the recipient's physiological system. In telemetry mode, the stimulations are delivered by means of a number of electrode stimulation channels. For example, the delivery of a stimulation current between two particular electrodes 142 of array 144 may be defined as a stimulation via channel 1. Similarly, other combinations of electrodes 142 involved in stimulation delivery will also define other stimulation channels. A telemetering arrangement is described in U.S. Pat. No. 5,758,651, the disclosure of which is hereby incorporated by reference herein.

In some cases, an extra-cochlear electrode arranged on the case of the implanted receiver unit 132 may be used as a reference electrode in measuring the evoked action potential of the auditory nerve. Alternatively or additionally an extra-cochlear electrode may be attached to the stapedius muscle to provide signals indicative of stapedius reflex activity.

Conventionally, a telemetry facility is used intra-operatively to test correct functioning of the implanted components of the cochlear implant system and auditory system function. However, in contrast with conventional telemetry systems, the telemetry system used to implement the present configuration method 250 is arranged to advantageously receive and process, in a manner to be described, multiple different types of physical measurements.

In one example, the present configuration method 250 determines a ‘dynamic range’ for each electrode stimulation channel, for the recipient-specific input configuration variables.

Next at block 253, one or more objective physical characteristics of the recipient's physiological system are measured by measurement module 165, to obtain corresponding objective physical measurement values 165A. Objective physical measurement values 165A are typically obtained by stimulating and then measuring a neural response arising under certain conditions, from the auditory nerve, auditory brain stem or higher regions of the recipient's auditory system, or the stapedius muscle.

Examples of such objective physical characteristics 165 used to obtain objective physical measurement values 165A include, for example:
ECAP threshold: the lowest stimulus current level at which an electrically evoked compound action potential (ECAP) is reliably observed. The unit used can be Current Level (CL) which is related to current flow according to Equation 1.

$$I ( μA ) = 17.5 \times {100}^{\frac{C L}{255}} \qquad ( Equation 1 )$$

As would be apparent to those skilled in the art, the exact form of Equation 1 will vary depending on the type of cochlear implant.
ECAP amplitude growth: the increase in peak-to-peak ECAP amplitude per increase in stimulus current level.
ECAP recovery: the duration of neural recovery after stimulus; for example, as determined from the slope of the ECAP amplitude curve, plotted with respect to time after stimulus.
ECAP spread of excitation: the width of stimulus spread; for example, as determined from the lateral distance of an alternative measuring electrode that results in a 25% reduction in ECAP amplitude (i.e. 75% of the ECAP amplitude that is obtained when the default measuring electrode is used).
EABR threshold: the lowest stimulus current level at which an electrically evoked auditory brain-stem response (EABR) is reliably observed.
ESR threshold: the lowest stimulus current level at which an electrically evoked stapedius reflex (ESR) is reliably observed.
Impedance: measured between a given intracochlear electrode and a reference electrode (such as an extra-cochlear electrode); impedance is influenced by both the electrode's physical status and the underlying tissue and fluid.

The above physical characteristics of the auditory system are described in detail in Cullington H. E., Cochlear Implants: Objective Measures, London, Whurr Publishers, 2003, whose disclosure is herein incorporated by reference in its entirety. As will be appreciated by those skilled in the art, this list of physical characteristics is not exhaustive and the use of other objective physical characteristics 165 in the configuration method 250 is envisaged.

At block 254, a number of stimulation parameter values 175A are optionally selected. The selection 170 of optional stimulation parameter values 175A is also shown in the data flow diagram of FIG. 2B. In this exemplary application of a cochlear implant, these stimulation parameter values 175A relate to the stimulation of the recipient's physiological system and measurement of the associated physical characteristics. In certain specific embodiments, stimulation parameter values 175A include stimulation rate and/or stimulation pulse width.

As would be apparent to those skilled in the art, variation of the stimulation rate and stimulation pulse width may influence the T and C levels (i.e. the input configuration variables 180A). The stimulation rate determines how many pulses are delivered per unit of time on any given electrode. Thus, if more pulses are included, less current per pulse is required, and accordingly the T and C levels will generally be lower. Similarly, a larger pulse width will typically imply lower T and C levels because the amplitude of each pulse should be less to maintain the same overall charge.

Generally however, default settings are employed for these stimulation parameter values and as such stimulation parameter values 175A such as stimulation rate and/or stimulation pulse width will not require selection as default values will be employed. Stimulation parameter values 175A, therefore, are not otherwise subjectively determined by a recipient and hence do not need to be measured.

The objective physical measurement values 165A measured at block 253 and the optional stimulation parameter values 175A selected at block 254, together form a set of input data for subsequent operations of configuration method 250.

Proceeding to block 255, the input data generated at blocks 253 and 254 is applied to a predictive configuration model 180 (also shown in FIG. 2B). The predictive configuration model 180 processes the input data (usually specific for each channel), and outputs values for each of the corresponding recipient-specific input configuration variables 180A. In this particular example, the recipient-specific input configuration variables 180A are a T level value and a C level value for each electrode/stimulation channel.

At block 256, the values of the input configuration variables 180A are stored in electronic memory and at block 257, the cochlear implant system returns to standard mode. In this example, configuration method 250 finishes at block 258.

Advantageously, configuration method 250 may be performed on a cochlear implant system 185 shortly after the surgical implantation procedure, thus allowing the recipient to immediately hear with the use of the implant upon regaining consciousness. In many cases, cochlear implant system 185 requires no further configuration, thus substantially reducing the need for post-operative clinical sessions. Alternatively, if further configuration is required, then the time required to do so is far less than the time required using conventional techniques.

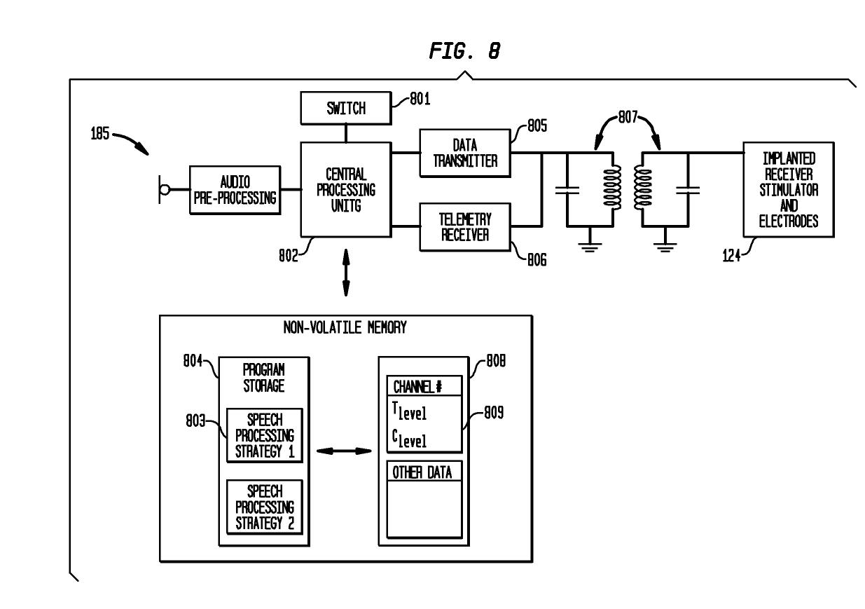

Turning now to FIG. 8, various operational and architectural aspects of cochlear implant system 185 incorporating the use of configuration method 250 will be described.

Upon pressing switch 801, a central processing unit (CPU) 802 retrieves an automatic configuration program 803 from program storage memory 804. CPU 802 then executed the automatic configuration program 803.

Automatic configuration program 803 initially places the cochlear implant system 185 into a telemetry mode, as noted in connection with block 252 of FIG. 2A. CPU 802 transmits code for a test stimulus pulse via a data transmitter 805 and transcutaneous link 807. This code includes information about which electrodes are to deliver the stimulation and the stimulation amplitude and duration, which are retrieved from the recipient data storage memory 808.

The received transmission signal is decoded by receiver unit 132 and the prescribed stimulation is applied to the implanted electrode array. The evoked action potential of the auditory nerve in response to the stimulation is monitored by receiver unit 132 and telemetered back to telemetry receiver 806 via transcutaneous link 807. This procedure is repeated several times and the recorded data is conditioned and tested for significance. This procedure is then repeated for all stimulation channels.

Automatic configuration program 803 then applies the predictive configuration model 180 to the input data as has been earlier described. At the conclusion of the automatic configuration program 803, the values of the recipient-specific input configuration variables are stored as entries in the recipient data storage T and C level table 809.

In this example, predictive configuration model 180 is directly programmed into the recipient's cochlear implant system 185. In this case, after objective physical measurements 165A are taken, the recipient-specific input configuration variables 180A can be automatically set for each electrode channel. Further, the recipient can, through a suitable control device, signal cochlear implant 185 to conduct measurements of the neural response of the cochlea and dynamically adjust the values of the recipient-specific input configuration variables 180A.

In another embodiment, the predictive configuration model 180 is programmed into a separate device that is used by the surgeon or clinician to calculate the relevant recipient-specific input configuration variables 180A, which are then uploaded into cochlear implant 185.

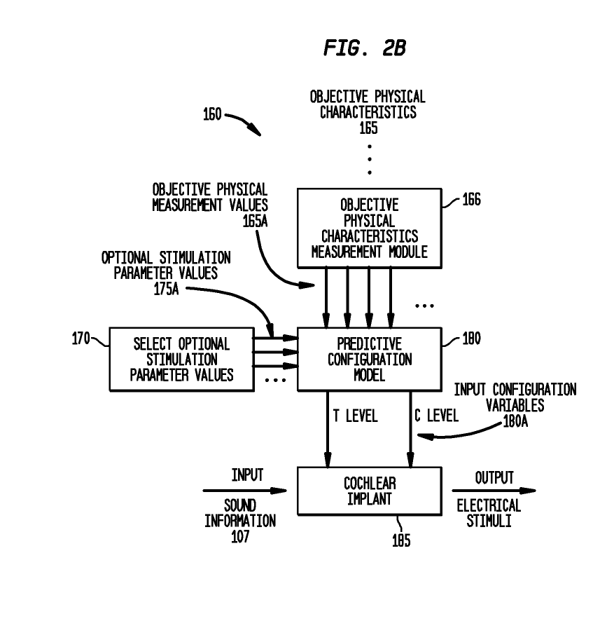

FIG. 3 is a schematic overview of the data flow used to create the predictive configuration model 180 (FIG. 2B) as used in the automatic configuration program 803 (FIG. 2B).

In this example, predictive configuration model 180 is based on data mining principles created from large sets of empirical data.

In this example, input training data 220 is used to build the predictive configuration model 180 includes many instances of individual sets of matching data, which have been empirically obtained from a large number of cochlear implant recipients. These individual sets of matching data may include objective physical measurements 210A, training configuration variables 220, and stimulation parameters 210B.

More specifically in this example, objective physical measurements 210A may include any one or more of the characteristics earlier identified, including, but not limited to ECAP threshold, ECAP amplitude growth, ECAP recovery, ECAP spread of excitation, EABR threshold, ESR threshold and/or impedance.

Similarly, the training configuration variables can include, but are not limited to subjectively determined T levels (Tₜᵣₐᵢₙ) for each channel, and subjectively determined C levels (Cₜᵣₐᵢₙ) for each channel.

As noted, optional stimulation parameters 210B may include, although are not limited to, stimulation rate and/or stimulation pulse width.

In overview, a machine learning algorithm 200 undergoes a “training” process to determine a wide range of predicted T and C levels (i.e., T_{pred} and C_{pred}) 230 for a corresponding wide range of subjectively determined T and C levels 220 (i.e., Tₜᵣₐᵢₙ and Cₜᵣₐᵢₙ)

Machine learning algorithm 200 uses the individual sets of matching data to make adjustments to an internal model, to eventually find associations or relationships between one or more of the following: objective physical measurements 210A; predicted configuration variables e.g., T and C levels (i.e. T_{pred} and C_{pred}) and stimulation parameters 210B e.g., map rate, stimulation pulse width, and the like.

Hence, after the training process, the predictive configuration model 180 created by machine learning algorithm 200 may be used to automatically calculate and provide T_{pred} and C_{pred} levels, based upon objective physical measurement values and optional stimulation parameter values such as stimulation rate and other settings.

Whilst in this embodiment, the automatic calculation of the input configuration variables is conducted on a channel by channel basis, equally the same process may be applied to groups of neighbouring channels. In this case, objective measurements for three channels and corresponding measured T and C levels for these three channels can be employed to train a machine learning algorithm that predicts the T and C levels for these same channels as a group.

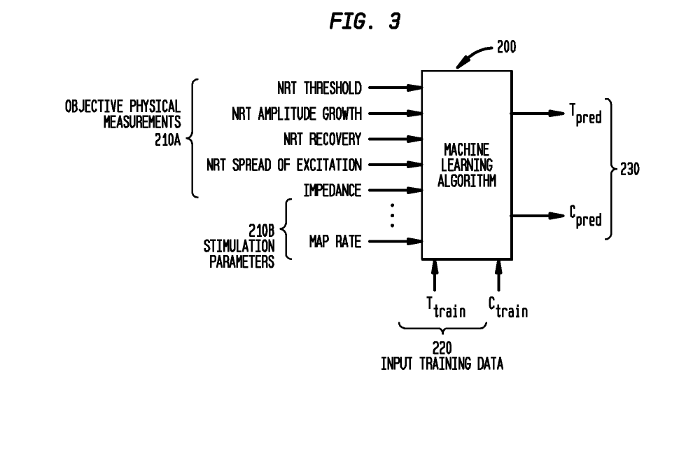

FIG. 4 is a subset of an exemplary data set used to train machine learning algorithm 200 according to an embodiment of the present invention. In this embodiment, a total of 84 records have been employed. Each record corresponds to a row of the table depicted in FIG. 4 and comprises of objective data measured for a given recipient (i.e. columns 310, 320, 330, 340, 350A, 350B, 360A, 360B, 360C), stimulation parameter values (370A, 370B) and the corresponding T levels (column 380) and C levels (column 390) as measured by a clinician. In this example any missing data is denoted by ‘?’ and is replaced by the measurement mean during the training process.

At column 310, “Electrode” is the sequential position of the stimulating electrode 142 in electrode array 144. At column 320, “Impedance (kOhm)” is the electrical impedance measured between the given stimulating electrode and a reference electrode (usually an extra-cochlear electrode). At column 330, “T-NRT (CL)” is the ECAP threshold as described previously. At column 340, “AGF (uV/CL)” is the ECAP amplitude growth as described previously.

At column 350A, “Recovery tau (μs)” is the curviness of the neural recovery function, as determined from the ECAP amplitude curve, plotted with respect to time after an initial masking stimulus. Larger tau indicates a slower rate of recovery (a shallower curve). At column 350B, “Recovery t0 (μs)” is the time intercept of the neural recovery function, as determined from the ECAP amplitude curve, plotted with respect to time after an initial masking stimulus. Larger t0 indicates a slower rate of recovery (a further time-shifted curve). At column 360A, “Total SOE (mm)” is the spread of excitation in both the basal and apical cochlear directions at a given stimulating electrode. At column 360B, “Apical SOE (mm)” is the spread of excitation in the apical cochlear direction at a given stimulating electrode. At column 360C, “Basal SOE (mm)” is the spread of excitation in the basal cochlear direction at a given stimulating electrode.

At column 370A, “MAP Rate (Hz)” is the number of biphasic pulses per second on each electrode that is used to deliver sound perception in a given cochlear implant configuration (i.e. the stimulation rate as previously described). At column 370B, “MAP Pulse width (μs)” is the width of each phase of each biphasic pulse that is used to deliver sound perception in a given cochlear implant configuration (i.e. the pulse width as previously described). At columns 380 and 390, “MAP T (CL)” and “MAP C (CL)” are the T and C levels as previously described in the specification.

In this exemplary embodiment, machine learning algorithm 200 is a rule-based predictive model created with the data mining tool Cubist produced by Rulequest Research Pty Ltd. Cubist employs a model tree approach to generate a predictor based on sets of linear functions of input values. The Cubist data mining tool is described in Quinlan J. R., An Overview of Cubist, Rulequest Research, http://www.rulequest.com/cubist-win.html and Quinlan J. R. (1992), Learning With Continuous Classes, Proceedings of the Fifth Australian Joint Conference on Artificial Intelligence (AI'92), pp. 343-348, Singapore: World Scientific, where both disclosures are herein incorporated by reference.

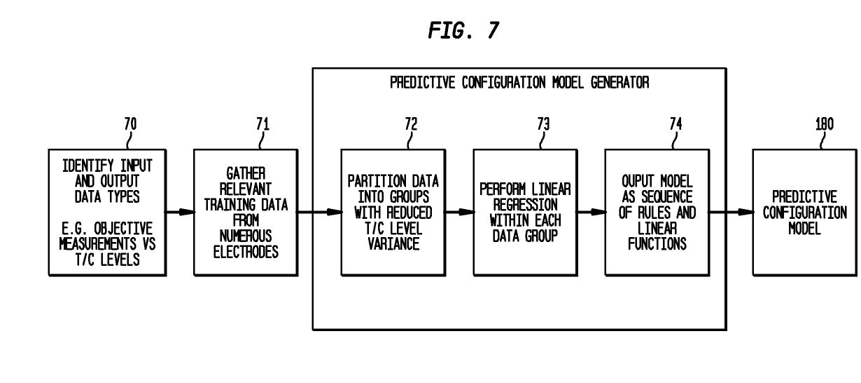

Referring to FIG. 7, the process of building a predictive configuration model using, for example, Cubist, includes a first block 70 at which the input and output data types are identified. Thereafter, at block 71, the input data are collected and fed into Cubist. At block 72, Cubist arranges the input data into groups, such that each group (specified by a rule, e.g. Rate <1200 Hz), contains reduced variances for each of the target variables.

Proceeding to block 73, Cubist performs linear regression within each group to specify a relation between the input data and the target variables. Next at block 74, Cubist outputs a predictive configuration model 180 as a sequence of rules and linear functions that is capable to predict T/C levels 180A based on input objective physical measurement values 165A (and optional stimulation parameter values) as illustrated in FIG. 2B.

Accordingly, in this exemplary embodiment, in order to predict the T_{pred} level based on the objective physical measurements the following calculation is performed.

If Rate is less than or equal to 1200 Hz then:

T_{pred}=43.5−0.0174 Rate−1.36 Pulse_Width+0.95T-NRT−3.8 Impedance−0.003 Recovery_tau+3 SOE_{—}75% Apical (Equation 2)

otherwise if Rate is greater than 1200 Hz then:

T_{pred}=18.3+0.9T-NRT−0.5 Pulse_Width−0.006 Recovery_tau+6 SOE_{—}75% Apical (Equation 3)

where:
T_{pred} is a current level (CL) value based on the relationship of Equation 1.
Rate is the stimulation rate in Hz as described previously;
Pulse_width is the pulse width of the stimulation signal in us as described previously;
T-NRT is the ECAP threshold measured in CL based on Equation 1 as described previously;
Impedance is the electrode impedance in kΩ as described previously;
Recovery_tau is the ECAP recovery in us as described previously; and
SOE_{—}75%_Apical is the apical ECAP spread of excitation in mm as described previously.

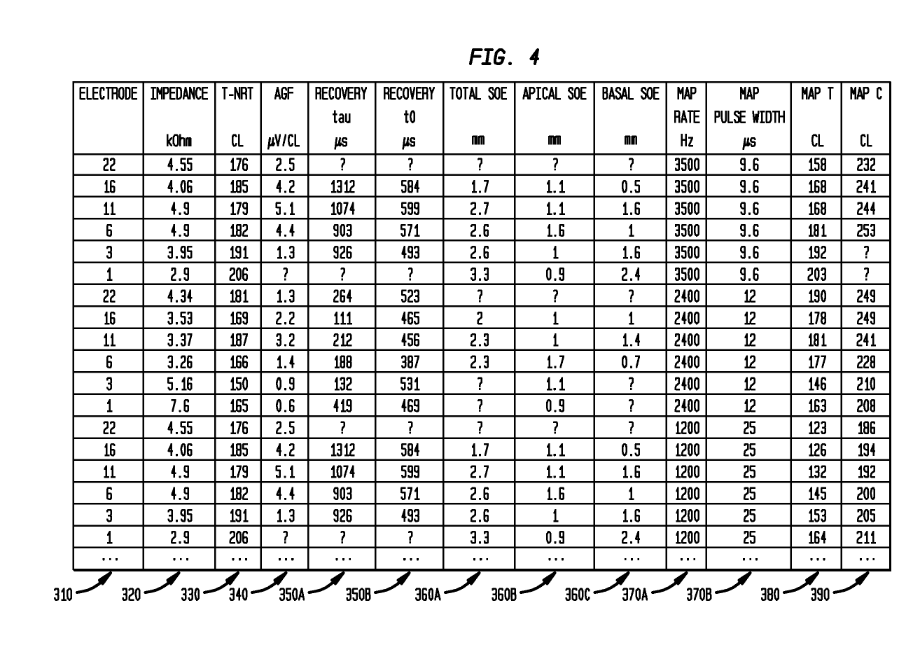

Referring now to FIG. 5, which is a graph of T_{pred} levels vs measured T levels, it can be readily appreciated that the predictive configuration model 180 provides a clinically useful T_{pred} level based on the input objective physical measurement values.

Turning now to the prediction of the C_{pred} level, the linear functions provided by Cubist allow C_{pred} to as t be calculated follows.

If Rate is less than or equal to 1200 Hz then:

C_{pred}=165−1.38 Pulse_Width+0.53T-NRT−5.9 Impedance−0.0052 Rate+0.37 Electrode (Equation 4)

otherwise if Rate is greater than 1200 Hz then:

C_{pred}=115.7+0.66T-NRT+0.0074 Rate−0.56 Pulse_Width−2.3 Impedance+0.12 Electrode (Equation 5)

where additionally Electrode is defined to be the position of the given electrode within the electrode array, numbered sequentially.

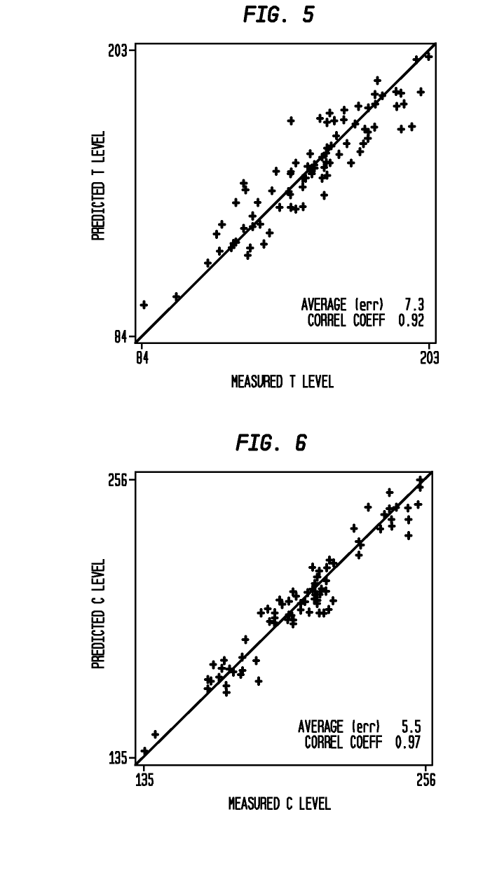

Referring now to FIG. 6, which is a graph of C_{pred} levels vs measured C levels it can be readily appreciated that the predictive configuration model 180 provides a clinically useful C_{pred} level based on the input objective physical measurements.

Predictive configuration model 180 may be created in other ways than the Cubist machine learning algorithm 200, for example, with the use of data analysis techniques such as multi-variate regression or other similar statistical techniques.

Further, the structure of the internal model can consist of neural networks, decision trees, rule lists, regression functions, or any combination of these structures as suitable, for example, Mitchell T., Machine Learning, New York, McGraw-Hill, 1997, or others. In general, the more training data available the greater the predictive power of machine learning algorithm 200 and hence the accuracy of the resultant predictive configuration model 180.

Further, predictive configuration model 180 may be based on a physiological model which directly simulates the auditory system to provide the T_{pred} and C_{pred} levels, based on the input objective physical measurements 165A.

The use of a predictive configuration model 180 in accordance with the various embodiments described herein enables multiple kinds and/or characteristics of objective physical measurement values of the recipient's auditory system to be used in determining the recipient-specific configuration variables. In particular, the normally subjectively determined T levels and C levels can now be more reliably correlated with objective physical measurements, thus reducing the need for subjective inputs from the recipient.

In other applications, recipient preferences for other additional input configuration variables that characterise the cochlear implant, such as the rate of loudness growth that is used to stimulate between T level and C level, can also be included and predicted.

The above described embodiments indicate that in the best case, an intra-operative configuration method can be performed, which is sufficient for the recipient and no post-operative expertise is required to further configure the cochlear implant. Alternatively, the recipient can be provided with an already pre-configured cochlear implant which will only require further tuning or configuration at their first post-operative clinical session, when given enough experience with his or her cochlear implant system to provide better feedback.

If predictive configuration model 180 is programmed directly into the cochlear implant, the recipient could periodically update his/her configuration variables such as the T and C levels, to adjust for any changes in the neural response of the auditory system caused by a change in the recipient's electrophysiological status in adaptation to the implanted device.

In this embodiment at the time of first use, following surgery, the cochlear implant system presents a map with C levels reduced to a conservative level to ensure safety. The recipient is free to then adjust C levels to establish a comfortable volume, but cannot do this beyond acceptable limits (for example, beyond 20% above the predicted levels).

In an alternative embodiment of the invention, the subjectively determined input configuration variables of the cochlear implant system can be used as inputs to the predictive configuration model, in addition to the aforementioned objective physical measurements and input stimulation parameters. This can be done after surgery, where the recipient is able to give an assessment of stimuli percepts. As an example of this alternative embodiment, subjectively determined T levels can be used as an input to a predictive configuration model that predicts C levels, or vice versa. This procedure still reduces the requirements of clinical sessions.

Whilst the present invention is described in relation to the configuration of a cochlear implant 185 it will be appreciated by those skilled in the art that the invention will have other applications consistent with the principles described in the specification.

For example, while cochlear implant system 185 is described as having external components, in an alternative embodiment the implant system 185 may be a totally implantable prosthesis in which speech processor 126, including the microphone and/or power supply, is implemented as one or more implantable components.

In other embodiments these methods and systems may be used with other implant systems such as, for example, in an auditory brain-stem implant or an electro acoustical device for a recipient. Also, it should be appreciated that although embodiments of the present invention are described herein in connection with implantable hearing devices, the same or other embodiments of the present invention may be implemented in other prosthetic devices as well. Examples of such devices include, but are not limited to, other sensory prosthetic devices, neural prosthetic devices, and functional electrical stimulation (FES) systems.

Those of skill in the art would appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Although a number of exemplary embodiments of the present invention have been described in the foregoing detailed description, it will be understood that the invention is not limited to the embodiment disclosed, but is capable of numerous rearrangements, modifications and substitutions without departing from the scope of the invention as set forth and defined by the following claims.

1. A method for configuring a medical implant that stimulates a physiological system of a recipient of the medical implant, the medical implant having at least one input configuration variable corresponding to a subjective characteristic of the physiological system, the method comprising:
measuring at least one physical characteristic of the physiological system to provide at least one objective physical measurement value; and
determining the at least one input configuration variable to configure the medical implant by a predictive configuration model having as an input the at least one objective physical measurement value.

2. The method as of claim 1, wherein the predictive configuration model is based on determined associations between the at least one objective physical measurement value of the physiological system and the at least one input configuration variable for the medical implant.

3. The method of claim 2, wherein the predictive configuration model includes as an input at least one stimulation input parameter value.

4. The method of claim 2, wherein the determined associations are determined by a machine learning algorithm trained on training data including multiple records of the at least one objective physical measurement value and corresponding at least one input configuration variable.

5. The method of claim 4, wherein multiple records of the corresponding at least one input configuration variable are subjectively determined by prior recipients of the medical implant.

6. The method of claim 4, wherein the training data further includes multiple corresponding records of the at least one stimulation input parameter value.

7. The method of claim 2, wherein the determined associations are determined by a physiological model of the physiological system.

8. The method of claim 1, wherein measuring the at least one physical characteristic and then configuring the implant occurs during a surgical implantation procedure.

9. The method of claim 8, wherein the medical implant is further configured by the recipient of the medical implant after the surgical implantation procedure.

10. The method of claim 1, wherein the medical implant is an implantable neural prosthesis for the treatment of hearing loss.

11. The method of claim 10, wherein the implantable neural prosthesis is a cochlear implant having an array of electrodes.

12. The method of claim 11, wherein the at least one input configuration variable includes T and C levels for at least one electrode in the array of electrodes.

13. The method of claim 11, wherein the at least one objective physical measurement value is obtained from measurements of one or more physical characteristics of the auditory system including:
ECAP threshold;
ECAP amplitude growth;
ECAP recovery;
ECAP spread of excitation;
EABR threshold;
ESR threshold; and
impedance for at least one electrode in the array of electrodes.

14. The method of claim 1, wherein the at least one stimulation parameter value includes the map rates and/or stimulation pulse widths for at least one electrode in the array of electrodes.

15. A medical implant system for the stimulation of a physiological system of a recipient of the medical implant, the medical implant system having at least one input configuration variable corresponding to a subjectively determined characteristic of the physiological system, the medical implant system including:
measurement means to measure at least one physical characteristic of the physiological system to provide at least one objective physical measurement value; and
data processing means for processing the at least one corresponding objective physical measurement value according to a predictive configuration model to provide the at least one input configuration variable to configure the medical implant system.

16. The medical implant system of claim 15, wherein the predictive configuration model is based on determined associations between the at least one objective physical measurement value of the physiological system and the at least one input configuration variable.

17. The medical implant system of claim 16, wherein the predictive configuration model includes as an input at least one stimulation input parameter value to configure the medical implant system.

18. The method of claim 16, wherein the determined associations are determined by a machine learning algorithm trained on training data including multiple records of the at least one objective physical measurement value and corresponding at least one input configuration variable.

19. The medical implant system of claim 18, wherein the multiple records of the corresponding at least one input configuration variable are subjectively determined by prior recipients of the medical implant.

20. The medical implant system of claim 18, wherein the training data further includes multiple corresponding records of the at least one stimulation input parameter value.

21. The medical implant system of claim 16, wherein the determined associations are determined by a physiological model of the physiological system.

22. The medical implant system of claim 15, wherein the measurement means measures the at least one physical characteristic during a surgical implantation procedure.

23. The medical implant system of claim 22, wherein the data processing means processes the at least one corresponding objective physical measurement value according to the predictive configuration model to provide the at least one input configuration variable to configure the medical implant system during the surgical implantation procedure.

24. The medical implant system of claim 23, wherein the medical implant system is further configured by a recipient of the medical implant after the surgical implantation procedure.

25. The medical implant system of claim 15, wherein the medical implant system is an implantable neural prosthesis for the treatment of hearing loss.

26. The medical implant system of claim 25, wherein the implantable neural prosthesis is a cochlear implant having an array of electrodes.

27. The medical implant system of claim 26, wherein the at least one input configuration variable includes T and C levels for at least one electrode in the array of electrodes.

28. The medical implant system of claim 26, wherein the at least one objective physical measurement value is obtained from measurements of one or more physical characteristics of the auditory system including:
ECAP threshold;
ECAP amplitude growth;
ECAP recovery;
ECAP spread of excitation;
EABR threshold;
ESR threshold; and
impedance for at least one electrode in the array of electrodes.

29. The method of claim 26, wherein the at least one stimulation parameter value includes the map rates and/or stimulation pulse widths for at least one electrode in the array of electrodes.
